(19) United States
(12) Patent Application Publication
(10) Pub. No.: US 2004/0193176 A1
(43) Pub. Date: Sep. 30, 2004

(54) Implant removal

(76) Inventors: Heinz Gerngross, Ulm (DE); Eric Bartsch, Arth (CH)

Correspondence Address: CHRISTIE, PARKER & HALE, LLP, P.O. BOX 7068, PASADENA, CA, 91109-7068, US

(21) Appl. No.: 10/777,370

(22) Filed: Feb. 12, 2004

(30) Foreign Application Priority Data
Feb. 12, 2003 (EP) 03 003 102.5

(51) Int. Cl. A61B 17/88

(52) U.S. Cl. 606/104

(57) ABSTRACT

The invention relates to an apparatus for implant removal having an anchoring element which can be connected to the implant and having a coupling member which is fastened to the anchoring element and has a, length such that a handling section of the coupling member can be placed at a position in the patient's body disposed remotely from the anchoring element connected to the implant.

The invention relates to an apparatus for implant removal.

Above all in operations for the care of bone fractures, minimum invasive techniques are increasingly being demanded for the introduction of implants. This demand is in particular being made in connection with the implanting of medullary nails.

In the implanting of medullary nails, a stage has now been reached where only very small skin incisions are necessary with a length from approximately 2 to 3 cm. It is a problem that currently this minimum invasion cannot be maintained if the implant should be removed again after the healing of the bone fracture.

For this purpose, the implant must be located in the bone and be connected to an instrument for the knocking out. In many cases, this requires a much larger access to the implant than on its implantation. This circumstance is above all difficult to understand from the view of the patient, since originally only a small scar resulted from the actual operation on the insertion of the implant and now has to be made hugely bigger on the removal of the implant.

It is therefore the object of the invention to provide a possibility to eliminate the problem described above as much as possible and to make do with skin incisions which are small as possible both on inserting and removing implants.

This object is satisfied by the features of claim 1, and in particular in that an apparatus is provided for the removal of the implant which includes an anchoring element, which can be connected to the implant, and a coupling member which is fastened to the anchoring element and which has a length such that a handling section of the coupling member can be placed at a position in the body of the patient disposed remotely from the anchoring element connected to the implant.

In accordance with the invention, the anchoring element, and thus the implant to be removed, can be located without problem using the coupling member, irrespective of where, and in particular at what depth, the anchoring element connected to the implant is positioned in the body. The handling section of the removal apparatus in accordance with the invention serves for this purpose and is placed in the body in a manner suitable for the respective application.

The length of the coupling member can advantageously be dimensioned such that the handling section can be placed directly under the skin of the patient. For the removal of the implant, the skin only has to be incised at the appropriate position with a very small incision in order to reach the handling section of the coupling member. The coupling member can then either serve as a guide which guides the operator through the tissue precisely to the anchoring element, and thus to the implant to be removed, or—in the case of an implant seated comparatively loosely—the implant can simply be pulled out of the respective bone and out of the patient's body by means of the coupling member.

The implant removal apparatus can be used particularly advantageously in conjunction with medullary nails which are used for the care of bone fractures. Such medullary nails are in practice usually implanted with the aid of a target device and have for this purpose a coupling section for the target device which in particular includes a thread and which is provided at the end of the implanting with a terminating element, in particular of a screw-like cap-like kind, to protect the coupling section against ingrowing tissue.

Provision can be made in accordance with the invention for this terminating or protective element, which can be connected to the implant, to serve as the anchoring element of the implant removal apparatus in accordance with the invention to which the coupling member is fastened.

In a preferred variant of the invention, the anchoring element has a plug screw which has an externally threaded section which can be screwed to an internally threaded section formed at the implant.

The coupling member can have an elongate form. The coupling member can in particular be of flexible design.

The coupling member can, for example, be a cable or a wire.

Provision can further be made for the coupling member to be made up of a plurality of individual load carriers. These load carriers can be woven or wound with one another to form the coupling member.

The coupling member can furthermore be manufactured from a biocompatible material. The coupling member can consist of a suture material. Specific material examples for the coupling member are titanium or steel.

Provision can furthermore be made for the coupling member to be fastened at its one end to the anchoring element and to have the handling section at its other end.

The handling section can be made as a loop, a sling or an eye of the coupling member.

The invention furthermore relates to an operating system having a plurality of implants, in particular of medullary nails, and having a plurality of apparatuses for the removal of the implant such as have been explained above and whose anchoring element can in each case be connected to at least one of the implants.

Further embodiments of the invention are recited in the dependent claims, in the description and in the drawing.

The invention will be described in the following by way of example with reference to the drawing. There are shown:

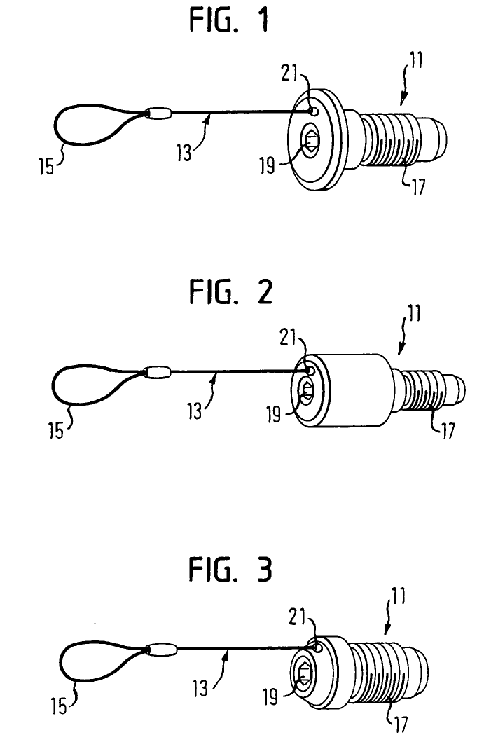

FIGS. 1-3 various embodiments of an apparatus for implant removal in accordance with the invention.

The embodiments shown in FIGS. 1-3 of an apparatus for implant removal in accordance with the invention differ from one another by the design of the anchoring element 11. The anchoring elements 11 are each matched to the respective implant (not shown).

In the embodiments shown, the anchoring elements 11 are in each case a plug screw which can be screwed to a fastening section of the implant having an internal thread in order to protect the fastening section or the internal thread from the tissue.

For this purpose, the anchoring elements 11 are each provided with an externally threaded section 17 as well as an actuation section 19 for a tool—here a hexagon socket.

The specific embodiment of the anchoring element 11 of the implant removal apparatus in accordance with the invention can generally be as desired. It is important that a reliable connection is possible between the anchoring element 11 and the respective implant and that a coupling member 13 can be fastened to the anchoring element 11. Said coupling member 13 will be looked at in more detail in the following.

The coupling element 13 in the embodiments shown is in each case an elongate flexible member in the form of a cable, of a-wire or of a band. While the one end of the coupling member 13 is fixedly connected at a joint 21 to the respective anchoring element 11, for example by welding, the free end of the coupling member 13 is formed as a sling or a loop 15 which forms a handling section of the implant removal apparatus in accordance with the invention. The loop or sling can be made such that it closes when pulled. Alternatively, the size of the loop or sling can also remain unchanged on the influence of pulling forces.

The handling section 15 of the coupling member 13 does not have to be formed as a sling or loop. The embodiment of the handling section 15 can generally be as desired and is selected according to the respective application.

The manner of the fastening of the coupling member 13 to the plug screw 11 can generally be as desired and is in particular selected in dependence on the materials used for the plug screw 11 and the coupling member 13. The type of the fastening is preferably selected such that relatively large pulling forces can also be transmitted to be able to pull out the implant connected to the plug screw 11 from the body of the patient via the coupling member 13 and the plug screw 11 at least in cases in which the implant is seated comparatively loosely

The flexible design of the coupling member 13 allows the sling 15 to be placed in a particularly simple manner at a selected position directly under the skin. For the removal of the implant, the skin can then be incised at the corresponding position, the coupling member 13 pulled tight and the tissue prepared obtusely along the coupling member 13 up to the bone when a pulling out of the implant via the coupling member 13, such as has been described above, is not possible.

An extensive search for the implant in the tissue becomes superfluous due to the possibility created in accordance with the invention for the localisation of the implant by means of the cable 13 serving as a guide in this respect, whereby only a very small incision in the skin is required which only needs to make possible the grasping of the sling 15 of the coupling member 13.

Consequently, thanks to the invention, the minimum invasive techniques advantageous for the patient can also be used in the implant removal.

In the embodiments described above, the invention advantageously uses the plug screw 11, which is anyway screwed to the respective implanted medullary screw, as an anchoring element which serves for the location and/or for the pulling out of the medullary nail and to which the coupling member 13 is fastened. However, in accordance with the invention it is not compulsory to use a component which is anyway present simultaneously as an anchoring element, but it is also possible to use anchoring elements manufactured directly for the purpose of the implant removal which are made such that they can be connected to the implant in a suitable manner.

Reference numeral list

11 anchoring element

13 coupling member

15 handling section

17 externally threaded section

19 actuation section

21 joint

1. An apparatus for implant removal comprising an anchoring element (11) which can be connected to the implant and comprising a coupling member (13) which is fastened to the anchoring element (11) and has a length such that a handling section (15) of the coupling member (13) can be placed at a position in a patient's body disposed remotely from the anchoring element (11) connected to the implant.

2. An apparatus in accordance with claim 1, wherein the anchoring element (11) can be screwed to the implant.

3. An apparatus in accordance with claim 1, wherein the anchoring element (11) is a terminal element for the protection of a fastening section, in particular of a thread section, of the implant usable for the insertion of the implant.

4. An apparatus in accordance with claim 1, wherein the anchoring element (11) is a plug screw and has an externally-threaded section (17) which can be screwed to an internally threaded section formed at the implant.

5. An apparatus in accordance with claim 1, wherein the coupling member (13) has an elongate shape.

6. An apparatus in accordance with claim 1, wherein the coupling member (13) is flexible.

7. An apparatus in accordance with claim 1, wherein the coupling member (13) is a cable.

8. An apparatus in accordance with claim 1, wherein the coupling member (13) is made up of a plurality of individual load carriers.

9. An apparatus in accordance with claim 1, wherein the coupling member (13) is a wire.

10. An apparatus in accordance with claim 1, wherein the coupling member (13) is manufactured from a biocompatible material.

11. An apparatus in accordance with claim 1, wherein the coupling member (13) is made of a suture material.

12. An apparatus in accordance with claim 1, wherein the coupling member (13) is manufactured from a material selected from the group consisting of titanium and steel.

13. An apparatus in accordance with claim 1, wherein the coupling member (13) is fastened at a first end to the anchoring element (11) and has the handling section (15) at a second end.

14. An apparatus in accordance with claim 13, wherein the handling section (15) is formed as a loop, a sling or an eye of the coupling member (13)

15. An apparatus in accordance with claim 1, wherein the length of the coupling member (13) is dimensioned such that the handling section (15) can be placed directly under skin of a patient.

16. An apparatus in accordance with claim 1, wherein it is made for the removal of medullary nails.

17. An operation system having a plurality of implants, in particular of medullary nails, and having a plurality of implant removal apparatuses in accordance with claim 1 whose anchoring element can in each case be connected to at least one of the implants.
